(19) United States
(12) Patent Application Publication
(10) Pub. No.: US 2026/0083334 A1
(43) Pub. Date: Mar. 26, 2026

(54) BLOOD VESSEL ESTIMATION DEVICE, BLOOD VESSEL ESTIMATION METHOD, AND PROGRAM

(71) Applicant: SHISEIDO COMPANY, LTD., Tokyo (JP)

(72) Inventors: Yuki TAKAHASHI, Tokyo (JP); Masato NINOMIYA, Tokyo (JP); Yuusuke HARA, Tokyo (JP); Takuma HOSHINO, Tokyo (JP); Masako KATSUYAMA, Tokyo (JP)

(21) Appl. No.: 19/112,668

(22) PCT Filed: Oct. 16, 2023
(86) PCT No.: PCT/JP2023/037441

§ 371 Date: Mar. 18, 2025

(30) Foreign Application Priority Data
Oct. 18, 2022 (JP) 2022-166887

(51) Int. Cl. A61B 5/02; A61B 5/00; A61B 5/0205; A61B 5/021

(52) U.S. Cl. CPC A61B 5/02007; A61B 5/0205; A61B 5/442; A61B 5/7267; A61B 5/7275; A61B 5/7278; A61B 5/021

(57) ABSTRACT

A condition of blood vessels is easily evaluated. One aspect of the present invention is a blood vessel estimation device including: an acquisition unit configured to acquire a skin index of a face and/or an internal index related to a skin of a subject; and a blood vessel estimation unit configured to estimate a capillary condition and/or a non-capillary blood vessel condition of the face of the subject from the skin index of the face and/or the internal index related to the skin of the subject.

### TECHNICAL FIELD

The present invention relates to a blood vessel estimation device, a blood vessel estimation method, and a program.

### BACKGROUND OF THE INVENTION

Conventionally, a method for visualizing blood vessels and evaluating the condition of blood vessels is known. For example, Patent Document 1 describes a method for acquiring an enlarged image from at least the upper dermis where microvessels are distributed, generating a blood vessel image by predetermined image processing for clarifying the microvessels in the acquired enlarged image, and evaluating at least one of the distribution and condition of the microvessels from the generated blood vessel image.

### RELATED-ART DOCUMENT

### Patent Document

Patent Document 1: Japanese Patent No. 5,977,408

### SUMMARY OF THE INVENTION

### Problems to be Solved by the Invention

However, conventional blood vessel visualization techniques require expensive specialized equipment for measurement and expensive machines for analysis. Therefore, there has been a demand for simple and low-cost evaluation of blood vessel conditions.

Therefore, an object of the present invention is to easily evaluate the condition of blood vessels.

### Means for Solving the Problems

In an embodiment of the present invention, a blood vessel estimation device includes: an acquisition unit configured to acquire a skin index of a face and/or an internal index related to a skin of a subject; and a blood vessel estimation unit configured to estimate a capillary condition and/or a non-capillary blood vessel condition of the face of the subject from the skin index of the face and/or the internal index related to the skin of the subject.

### Effects of the Invention

According to the present invention, it is possible to easily evaluate the condition of blood vessels.

### BRIEF DESCRIPTION OF THE DRAWINGS

FIG. 1 is an overall configuration example according to an embodiment of the present invention.

FIG. 2 is a functional block diagram of a blood vessel estimation device according to an embodiment of the present invention.

FIG. 3 is a flowchart of a blood vessel estimation process according to an embodiment of the present invention.

FIG. 4 is a diagram for explaining a regression model according to an embodiment of the present invention.

FIG. 5 is a diagram for explaining a regression model according to an embodiment of the present invention.

FIG. 6 is a diagram for explaining a regression model according to an embodiment of the present invention.

FIG. 7 is a diagram for explaining a regression model according to an embodiment of the present invention.

FIG. 8 is a hardware configuration diagram of a blood vessel estimation device and a terminal according to an embodiment of the present invention.

### DETAILED DESCRIPTION OF THE INVENTION

Hereinafter, an embodiment of the present invention will be described based on the drawings.

### Overall Configuration Example

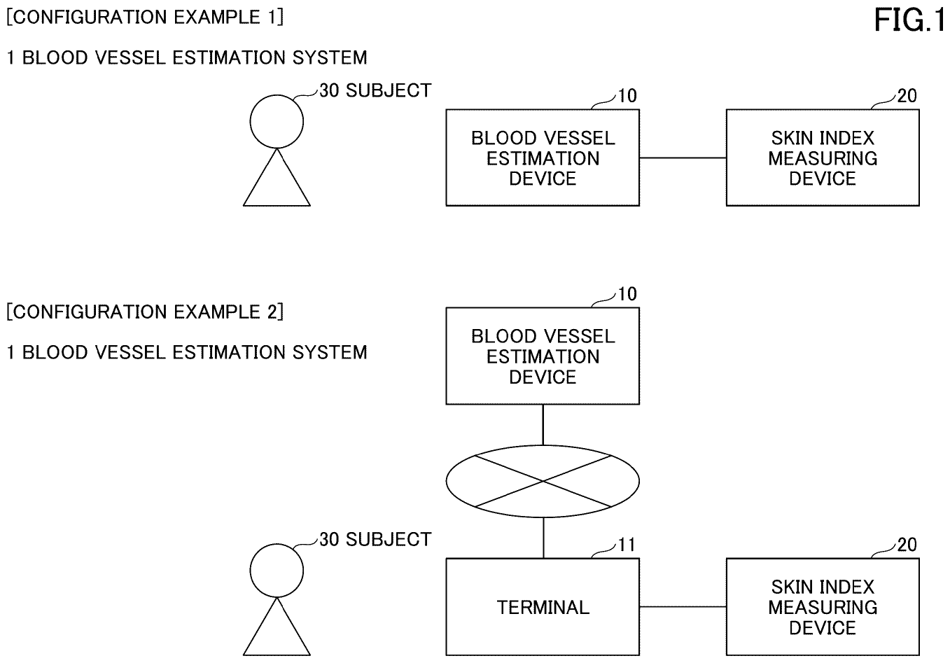

FIG. 1 is an overall configuration example according to an embodiment of the present invention.

### Configuration Example 1

A blood vessel estimation system 1 includes a blood vessel estimation device 10 and a skin index measuring device 20. Each of these devices will be described below.

### Blood Vessel Estimation Device

The blood vessel estimation device 10 is a device for estimating condition of capillaries (also referred to as capillary condition) and condition of blood vessels other than the capillaries (also referred to as non-capillary blood vessel condition) of a subject 30 (hereinafter, “capillaries” and “blood vessels other than capillaries” are collectively referred to as skin blood vessels). The blood vessel estimation device 10 may acquire the results measured by the skin index measuring device 20 from the skin index measuring device 20. For example, the blood vessel estimation device 10 is a personal computer, a tablet terminal, a smartphone, or the like.

Specifically, the blood vessel estimation device 10 acquires a skin index of a face of the subject 30. Also, the blood vessel estimation device 10 estimates the capillary condition and/or the non-capillary blood vessel condition (in other words, at least one of “the capillary condition” or “the non-capillary blood vessel condition”) of the face of the subject 30 from the skin index of the face of the subject 30. Also, the blood vessel estimation device 10 estimates which of a plurality of types future skin condition of the subject 30 is, from the capillary condition and/or the non-capillary blood vessel condition (in other words, at least one of “the capillary condition” or “the non-capillary blood vessel condition”) that are estimated. Also, the blood vessel estimation device 10 proposes (for example, displays on a screen of the blood vessel estimation device 10 or the skin index measuring device 20 (for example, a smart mirror) ) at least one of products or services related to beauty according to at least one of: the capillary condition and/or the non-capillary blood vessel condition (in other words, at least one of “the capillary condition” or “the non-capillary blood vessel condition”); or the future skin condition.

### Skin Index Measuring Device

The skin index measuring device 20 measures the skin index of the face and an internal index related to the skin of the subject 30. For example, the skin index measuring device 20 is a device capable of measuring at least one of the following: a blemish amount, transepidermal water loss (TEWL), redness, yellowness, a skin furrow area ratio, an unevenness of skin texture, a pore area ratio, collagen density, a wrinkle amount, a firmness index, a sagging degree, inflammatory cytokines, or blood pressure. For example, the skin index measuring device 20 is a device capable of measuring and analyzing skin indices, such as an imaging device (for example, a smart mirror (specifically, a mirror-type display with a built-in camera) ), or a horny layer analyzer. (In this case, at least one of the following and the like is acquired from information on a face (for example, an image of a face) of the subject 30 measured by the measuring device: a blemish amount, transepidermal water loss (TEWL), redness, yellowness, a skin furrow area ratio, an unevenness of skin texture, a pore area ratio, collagen density, a wrinkle amount, a firmness index, a sagging degree, or inflammatory cytokines. ) For example, the skin index measuring device 20 is a sphygmomanometer for measuring blood pressure.

### configuration Example 2

The blood vessel estimation system 1 includes the blood vessel estimation device 10, a terminal 11, and the skin index measuring device 20. The blood vessel estimation device 10 and the terminal 11 are communicably connected via an arbitrary network. Each of them will be described below.

### Blood Vessel Estimation Device

The blood vessel estimation device 10 is a device for estimating the capillary condition and the non-capillary blood vessel condition of the subject 30. The blood vessel estimation device 10 may acquire the results measured by the skin index measuring device 20 from the terminal 11. For example, the blood vessel estimation device 10 is a server or the like consisting of one or more computers.

Specifically, the blood vessel estimation device 10 acquires the skin index of the face of the subject 30. Also, the blood vessel estimation device 10 estimates a condition of the skin blood vessels, that is, the capillary condition and/or the non-capillary blood vessel condition, of the face of the subject 30 from the skin index of the face of the subject 30. Also, the blood vessel estimation device 10 estimates which of a plurality of types future skin condition of the subject 30 is, from the capillary condition and/or the non-capillary blood vessel condition that are estimated. Also, the blood vessel estimation device 10 proposes (for example, displays on a screen of the terminal 11 or the skin index measuring device 20 (for example, a smart mirror) ) at least one of products or services related to beauty according to at least one of: the capillary condition and/or the non-capillary blood vessel condition; or the future skin condition.

### Terminal

The terminal 11 acquires the skin index of the face of the subject 30 from the skin index measuring device 20, and provides the acquired skin index of the face of the subject 30 to the blood vessel estimation device 10. Also, the terminal 11 acquires from the blood vessel estimation device 10 and displays information on at least one of the products or services related to beauty according to at least one of: the capillary condition and/or the non-capillary blood vessel condition; or the future skin condition. The terminal 11 may execute a part of the processing of the blood vessel estimation device 10 described in the present specification. For example, the terminal 11 is a personal computer, a tablet terminal, a smartphone, or the like.

### Skin Index Measuring Device

Because the skin index measuring device 20 is the same as that of the configuration example 1, description thereof will be omitted.

In the configuration example 2, the blood vessel estimation system 1 be configured without the terminal 11, and the skin index measuring device 20 may directly transmit and receive data with the blood vessel estimation device 10.

### Functional Block

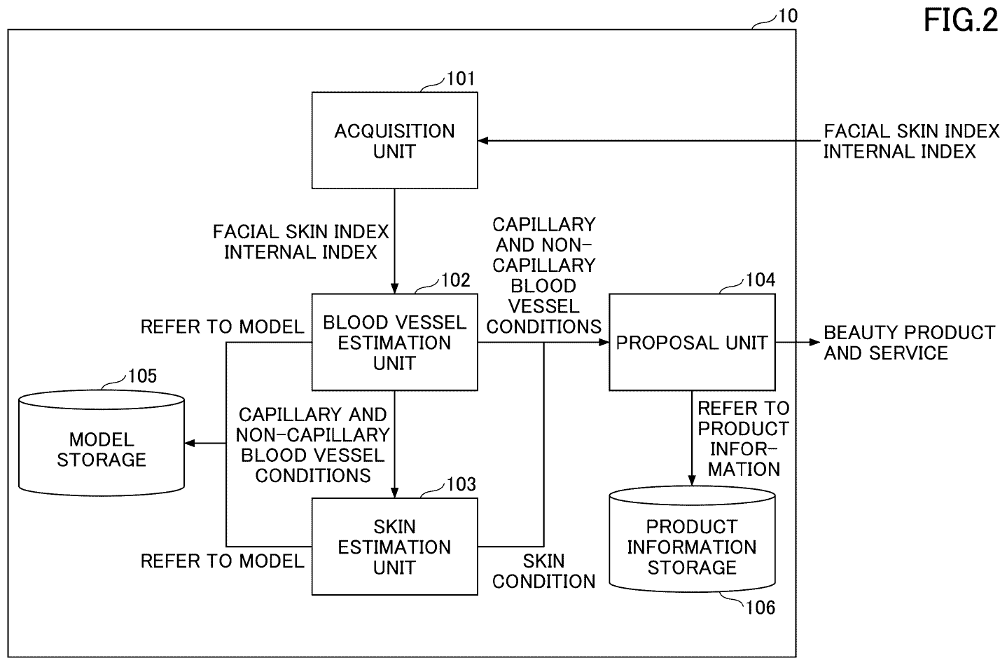

FIG. 2 is a functional block diagram of the blood vessel estimation device 10 according to an embodiment of the present invention. The blood vessel estimation device 10 includes an acquisition unit 101, a blood vessel estimation unit 102, a skin estimation unit 103, a proposal unit 104, a model storage 105, and a product information storage 106. The blood vessel estimation device 10 functions as the acquisition unit 101, the blood vessel estimation unit 102, the skin estimation unit 103, and the proposal unit 104, by executing a program. Each of these functions will be described below.

The acquisition unit 101 acquires the skin index and/or the internal index related to the skin (in other words, at least one of “the facial skin index” or “the internal index related to the skin”) of the face of the subject 30.

### When Using Index Measured by Skin Index Measuring Device 20

For example, the acquisition unit 101 may acquire the skin index of the face and/or the internal index related to the skin of the subject 30 measured by the skin index measuring device 20.

### When Using Index Acquired From Image Captured by Skin Index Measuring Device 20

For example, the acquisition unit 101 may acquire the skin index of the face of the subject 30 acquired from an image of the face of the subject 30 captured by the skin index measuring device 20 (for example, an imaging device).

### When Using Index Input by Subject 30 etc.

For example, the acquisition unit 101 may acquire the skin index of the face of the subject 30 input by the subject 30 or the like to the blood vessel estimation device 10 or to the terminal 11.

### Skin Index

The skin index will be described. The skin index is a skin index that can affect the condition of blood vessels.

For example, the skin index is an index related to skin aging. For example, the index related to skin aging is: a blemish amount, collagen density, a wrinkle amount, a firmness index, and a sagging degree.

For example, the skin index is an index related to skin roughness. For example, the index related to skin roughness is: transepidermal water loss (TEWL), redness, an unevenness of skin texture, and inflammatory cytokines.

For example, the skin index is at least one of the following: a blemish amount, transepidermal water loss (TEWL), skin tone (redness and yellowness), a skin furrow area ratio, an unevenness of skin texture, a pore area ratio, collagen density, a wrinkle amount, a firmness index, a sagging degree, or inflammatory cytokines. The information on the skin of the subject 30 acquired by the acquisition unit 101 may be the skin index only, or may be the skin index and the internal index related to the skin (for example, blood pressure). Each of them will be described below.

The “blemish amount” refers to the amount, color, area, and distribution of blemishes on the face.

The “transepidermal water loss (TEWL) ” refers to the amount of water evaporates through the horny layer.

The “redness” refers to the degree to which the skin is red.

The “yellowness” refers to the degree to which the skin is yellow.

The “skin furrow area ratio” refers to the percentage of the area of skin furrows per unit area of the face.

The “unevenness of skin texture” refers to the degree to which skin texture (irregularities on the skin surface) is uneven.

The “pore area ratio” refers to the percentage of the area of pores per unit area of the face.

The “collagen density” refers to the percentage of collagen per specific unit of the face, and the fineness of the collagen network.

The “wrinkle amount” refers to the number, the number, depth, area, and distribution of wrinkles.

The “firmness index” refers to the responsiveness of the skin when pressure is applied to the skin.

The “sagging degree” refers to the degree of swelling and sagging of the skin of the face.

The “inflammatory cytokines” includes cytokines collected from the horny layer, and refers to the amount of IL-1α, the amount of IL-1ra, the ratio of IL-1α to IL-1ra, the amount of SCCA1, and the amount of S100A8A9.

As described above, the skin index (for example, at least one of: the blemish amount, the transepidermal water loss (TEWL), the redness, the yellowness, the skin furrow area ratio, the unevenness of skin texture, the pore area ratio, the collagen density, the wrinkle amount, the firmness index, the sagging degree, or the inflammatory cytokines) may be acquired from an image the face of the subject 30 captured by the skin index measuring device 20. The blood vessel estimation device 10 (or the skin index measuring device 20) may calculate the skin index by analyzing the face image of the subject 30 captured by the skin index measuring device 20. The blood vessel estimation device 10 (or the skin index measuring device 20) may calculate, for example, the inflammatory cytokines by analyzing the collected horny layer. Furthermore, the blood vessel estimation device 10 (or the skin index measuring device 20) may calculate the internal index by measuring the blood pressure of the subject 30 with a sphygmomanometer.

The blood vessel estimation unit 102 estimates the capillary condition and/or the non-capillary blood vessel condition of the face of the subject 30 from the skin index of the face and/or the internal index related to the skin of the subject 30 acquired by the acquisition unit 101. Specifically, the blood vessel estimation unit 102 estimates the capillary condition and/or the non-capillary blood vessel condition of the face of the subject 30 from the skin index of the face and/or the internal index related to the skin of the subject 30, by using a regression model. The regression model may be a statistical model (that is, a mathematical model that derives the capillary condition and/or the non-capillary blood vessel condition of the face from the skin index of the face and/or the internal index related to the skin) or a machine learning model (that is, a trained model that has been trained using machine learning to output the capillary condition and/or the non-capillary blood vessel condition of the face when the skin index of the face and/or the internal index related to the skin is input).

Capillary Condition and Non-Capillary Blood Vessel Condition

Here, the capillary condition and the non-capillary blood vessel condition will be described. The capillary condition and/or the non-capillary blood vessel condition may be a principal component (also referred to as a blood vessel score) obtained by reducing the variables indicating the capillary condition and/or the non-capillary blood vessel condition by principal component analysis, or may be a variable itself indicating the capillary condition and/or the non-capillary blood vessel condition.

### Blood Vessel Score

For example, the capillary condition and the non-capillary blood vessel condition are the blood vessel score (the principal component obtained by reducing the variables indicating the capillary condition and the non-capillary blood vessel condition by principal component analysis), which is an objective variable of the regression model described later. The principal component correlates with at least one of (1) a density of blood vessels, (2) a number of the blood vessels (a number of capillaries and a number of large blood vessels), (3) a number of branches of the blood vessel, (4) a distance from the base of the epidermis to the blood vessels, or (5) a thickness of the epidermis.

### Capillary Condition Itself and Non-Capillary Blood Vessel Condition Itself

For example, the capillary condition and the non-capillary blood vessel condition are the capillary condition itself and the non-capillary blood vessel condition itself (that is, the capillary condition per se and the non-capillary blood vessel condition per se), which are objective variables of the regression model described later. Specifically, the capillary condition and the non-capillary blood vessel condition are at least one of: (1) the density of blood vessels, (2) the number of the blood vessels (the number of capillaries and the number of large blood vessels), (3) the number of branches of the blood vessels, (4) the distance from the base of the epidermis to the blood vessels, or (5) the thickness of the epidermis, itself (per se).

Each of these is described below.

The “number of blood vessels” refers to the number of capillaries and/or blood vessels other than the capillaries in the face. The number of capillaries refers to the number of blood vessels that are 40 μm or less in diameter, in which small blood vessels are included in addition to generally-known capillaries.

The number of large blood vessels refers to the number of blood vessels that are 160 μm or more in diameter among blood vessels other than the capillaries.

The “density of blood vessels” refers to the percentage of the capillaries and/or the blood vessels other than the capillaries per specific unit at the acquisition site of the face.

The “number of branches of blood vessels” refers to the number of branches of the capillaries and/or the blood vessels other than the capillaries.

The “distance from the base of the epidermis to blood vessels” refers to a representative value such as the mean value, the maximum value, the minimum value, and the median value of the distance between the base of the epidermis and the capillaries and/or the blood vessels other than the capillaries.

The “thickness of the epidermis” refers to a representative value such as the mean value, the maximum value, the minimum value, and the median value of the thickness of the epidermis.

The skin estimation unit 103 estimates which of a plurality of types the future skin condition of the subject 30 is, from the capillary condition and/or the non-capillary blood vessel condition of the face of the subject 30 estimated by the blood vessel estimation unit 102. Specifically, the skin estimation unit 103 estimates which of a plurality of types the future skin condition of the subject 30 is, from the capillary condition and/or the non-capillary blood vessel condition of the face of the subject 30, by using a model. The model may be a statistical model (that is, a mathematical model that derives which of a plurality of types the future skin condition is, from the capillary condition and/or the non-capillary blood vessel condition of the face) or a machine learning model (that is, a trained model that has been trained using machine learning to output which of a plurality of types the future skin condition is when the capillary condition and/or the non-capillary blood vessel condition of the face are input).

The proposal unit 104 proposes at least one of products or services related to beauty according to at least one of: the capillary condition and/or the non-capillary blood vessel condition estimated by the blood vessel estimation unit 102; or the future skin condition estimated by the skin estimation unit 103. Specifically, the proposal unit 104 proposes at least one of products or services related to beauty according to at least one of: the capillary condition and/or the non-capillary blood vessel condition; or the future skin condition, based on the correspondence between at least one of the capillary condition and/or the non-capillary blood vessel condition or the type of the future skin condition and at least one of the products or services related to beauty. It is assumed that the correspondence is determined in advance.

For example, the products related to beauty are basic cosmetics such as lotions, milky lotions, beauty lotions, and creams. For example, the products related to beauty are beauty equipment, food and drink such as supplements and beverages, and the like.

For example, the services related to beauty are beauty treatments, beauty consulting, and the like.

The model storage 105 stores the model referred to by the blood vessel estimation unit 102 and the model referred to by the skin estimation unit 103.

The product information storage 106 stores the correspondence referred to by the proposal unit 104 (specifically, the correspondence between at least one of the capillary condition and/or the non-capillary blood vessel condition (that is, the condition of the skin blood vessels) or the type of the future skin condition and at least one of the products or services related to beauty suitable for people with at least one of the capillary condition and/or the non-capillary blood vessel condition (that is, the condition of the skin blood vessels) or the type of the future skin condition).

### Method

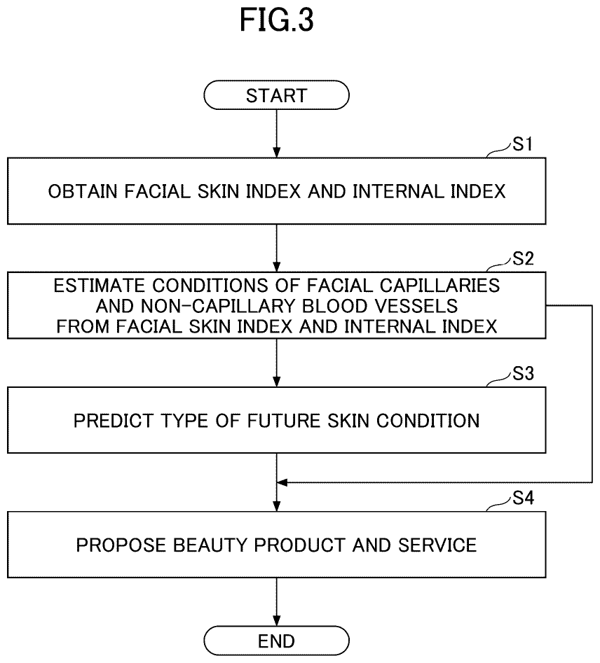

FIG. 3 is a flowchart of a blood vessel estimation process according to an embodiment of the present invention.

In step 1 (S1), the blood vessel estimation device 10 (the acquisition unit 101) acquires the skin index of the face and/or the internal index related to the skin of the subject 30.

In step 2 (S2), the blood vessel estimation device 10 (the blood vessel estimation unit 102) estimates the capillary condition and/or the non-capillary blood vessel condition (that is, the condition of the skin blood vessels) of the face of the subject 30, from the skin index of the face and/or the internal index related to the skin of the subject 30 acquired in step S1.

In step 3 (S3), the blood vessel estimation device 10 (the skin estimation unit 103) estimates which of a plurality of types the future skin condition of the subject 30 is, from the capillary condition and/or the non-capillary blood vessel condition of the face of the subject 30 estimated in step S2.

In step 4 (S4), the blood vessel estimation device 10 (the proposal unit 104) proposes at least one of the products or services related to beauty according to at least one of the capillary condition and/or the non-capillary blood vessel condition estimated in step S2 or the future skin condition estimated in step S3.

### Generation of Regression Model

Hereinafter, the generation of the regression model used in the present invention for estimating the capillary condition from the skin index of the face will be described.

### Objective Variable (Capillary Condition)

In the present invention, data obtained by an optical coherence tomography system (OCT) were subjected to principal component analysis, and a principal component calculated by the principal component analysis was used as an objective variable of the regression model. Specifically, image analysis was performed on images captured by the OCT to evaluate the three-dimensional structure of the vascular network in the dermis. Subsequently, quantitative analysis of the vascular network in the dermis was performed (the quantitative items were (1) the density of blood vessels, (2) the number of the blood vessels (the number of capillaries and the number of large blood vessels), (3) the number of branches of the blood vessels, (4) the distance from the base of the epidermis to the blood vessels, and (5) the thickness of the epidermis), and the quantitative values of the blood vessels were reduced by the principal component analysis, and the relationship between the blood vessels and the skin was analyzed.

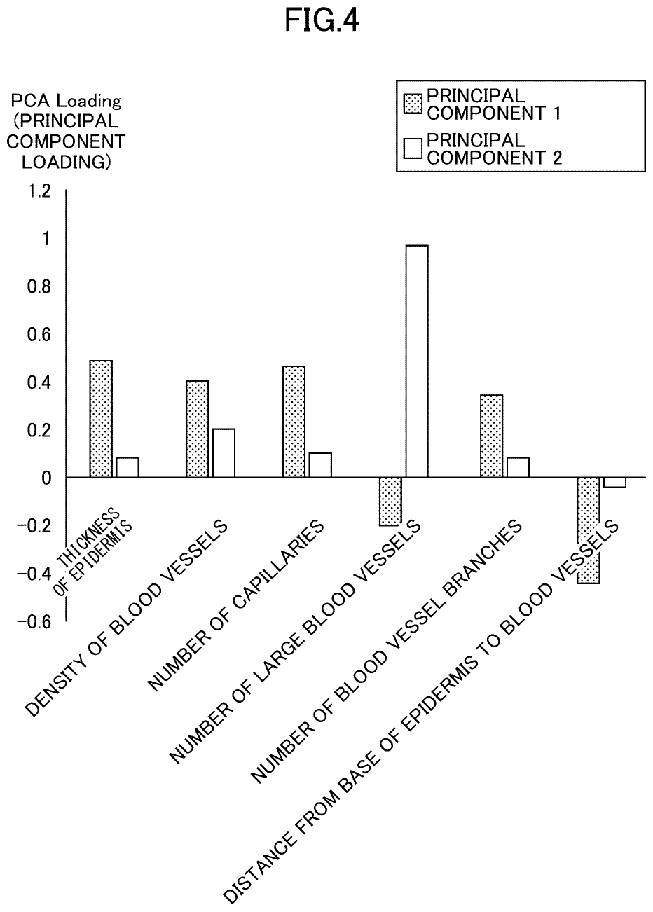

FIG. 4 illustrates the results of the principal component analysis. The principal component loadings of two variables (a principal component 1 (PC1) and a principal component 2 (PC2) ) generated by the principal component analysis are indicated.

The PC1 was positively correlated with “the number of capillaries”, “the density of blood vessels”, “the number of branches of the blood vessels”, and “the thickness of the epidermis”. The PC1 was negatively correlated with “the distance from the base of the epidermis to the blood vessels”. In the present invention, the PC1 was used as the objective variable of the regression model. (The PC1 indicates how tightly the small blood vessels are networked. The PC1 is also referred to as the blood vessel score.) The PC2 was positively correlated with “the number of large blood vessels”.

### Model Generation

A regression model for estimating the PC1 (the blood vessel score) was examined using multiple regression analysis. Specifically, combinations of explanatory variables were examined in a brute-force manner to search for significant explanatory variables (the skin index of the face and the internal index related to the skin).

### Measurement of Skin Index of Face

First, the values of the following measurement items were measured for the following subjects. All measurement values were standardized (mean: 0, variance: 1).

### Subjects

gender: female, number of subjects: 123, age: 20 s-70 s

### Measurement Items:

age, moisture content, transepidermal water loss (TEWL), collagen density, wrinkle amount, firmness index, sagging degree, unevenness of skin texture, skin furrow area ratio, pore area ratio, skin tone (redness and yellowness), blemish amount, inflammatory cytokines, and advanced glycation end products (AGEs) amount

The PC1 (the blood vessel score) was calculated for the above subjects.

Next, the flow of the multiple regression analysis is explained.

The following steps were performed in the following order.
1. reducing explanatory variables to avoid multicollinearity
2. constructing a multiple regression model with all combinations of the explanatory variables
3. selecting model candidates based on the akaike information criterion (AIC)
4. determining an optimal model based on the significance of the regression coefficients of the explanatory variables
   The steps 1. to 4. will be described in detail below.

### 1. Reducing of Explanatory Variables

Variables were reduced so that the value inflation factor (VIF) value did not exceed a certain value, eliminating the effect of multicollinearity.

### 2. Creating of List of Multiple Regression Models

A multiple regression model was constructed (by using the least squares method) with all combinations of the explanatory variables, and the AIC was calculated to evaluate the model.

Here, the AIC is explained. The AIC is an index of the goodness of fit of a model, and the smaller the AIC, the better.

### 3. Selecting of Group of Model Candidates

Optimal model candidates were selected based on the value of the AIC value, interpretability, and variable dominance.

### 4. Selecting of Model with Significant Variables

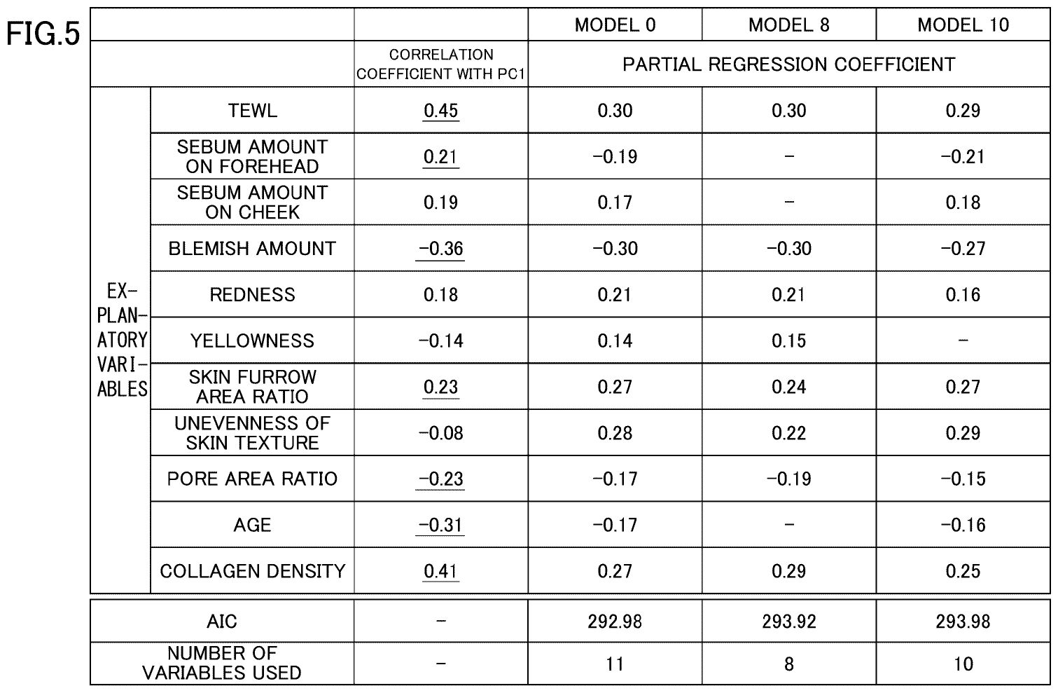

A model in which all variables were significant was searched for, with a p-value of 0.1 as the criterion. FIG. 5 indicates three models in which all variables were significant.

Because a model 8 can explain the PC1 (the blood vessel score) with a small number of variables, it can be said that the model 8 has high interpretability. In addition, in the model 8, for the variables correlated with the PC1 (the blood vessel score) (correlation of |r|>0.2 or more), because the coefficients of simple correlation and partial regression are consistent in whether they are positive or negative, it can be said that the validity of the model 8 is high. The correlation coefficient (Spearman's rank correlation coefficient) and VIF value between the explanatory variables in the model 8 were kept low, and it was determined that the risk of multicollinearity was low.

In an embodiment of the present invention, a model 10 or a model 0 may be used.

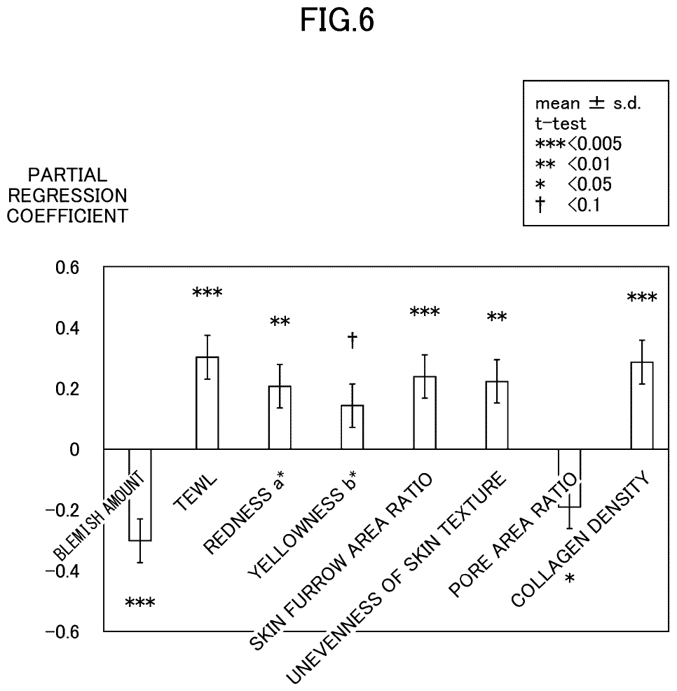

FIG. 6 illustrates explanatory variables contributing to the PC1 (the blood vessel score) of the model 8 and their interpretation. By summarizing the factors necessary for estimating the PC1 (the blood vessel score), it was inferred that the two factors of “aging” and “skin roughness” are related. Specifically, it was found that when there is a tendency for skin aging (having more blemishes and lower collagen levels), the PC1 (the blood vessel score) becomes lower. It was found that when there is a tendency for skin roughness (having higher TEWL and redness, and skin texture being more disturbed), the PC1 (the blood vessel score) becomes higher. The results in FIG. 6 suggest that the PC1 (the blood vessel score) can be estimated by the following equation.

$$PC 1 ( the blood vessel score ) = - 0.3 \times blemish amount + 0.3 \times TEWL + 0.21 \times redness + 0.15 \times yellowness + 0.24 \times skin furrow area ratio + 0.22 \times unevenness of skin texture + - 0.19 \times pore area ratio + 0.29 \times collagen density$$

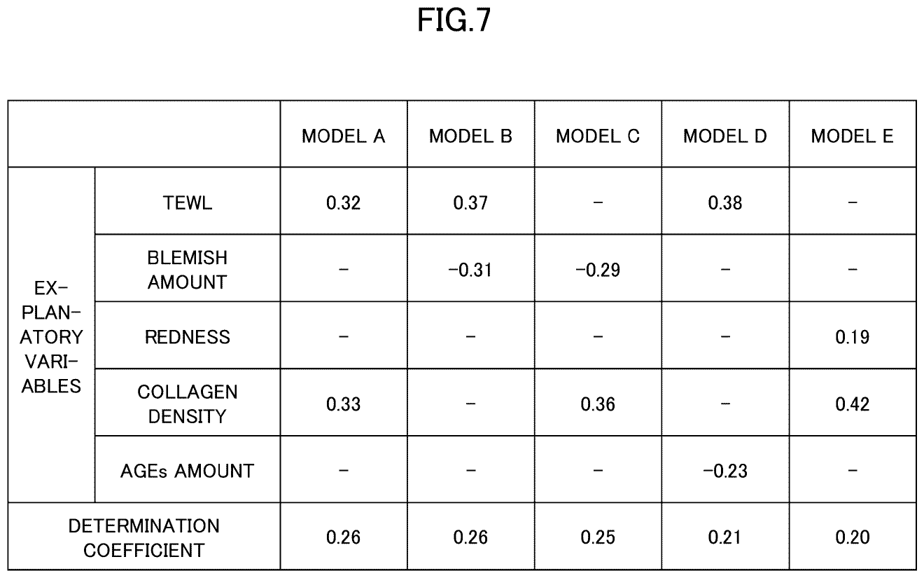

FIG. 7 illustrates a list of the smallest models for estimating capillary condition. The explanatory variables and coefficients for each model (a model A to a model E) are listed. In the model A, PC1 (the blood vessel score)=0.32×TEWL+0.33×collagen density, and the determination coefficient is 0.26. In the model B, PC1 (the blood vessel score)=0.37×TEWL+−0.31×blemish amount, and the determination coefficient is 0.26. In the model C, PC1 (the blood vessel score)=−0.29×blemish amount+0.36×collagen density, and the determination coefficient is 0.25. In the model D, PC1 (the blood vessel score)=0.38×TEWL+−0.23×AGES amount, and the determination coefficient is 0.21. In the model E, PC1 (the blood vessel score)=19×redness+0.42×collagen density, and the determination coefficient is 0.20.

In an embodiment of the present invention, the above-described model 8 with high estimation accuracy may be used (the model 10 or the model 0 may be used), or the above-described models A to E with fewer explanatory variables may be used.

### Other Embodiments of Generation of Regression Model

Hereinafter, other embodiments of the generation of the regression model used in the present invention for estimating the capillary condition or the non-capillary blood vessel condition from the skin index of the face will be described.

### Objective Variable (Capillary Condition or Non-Capillary Blood Vessel Condition)

(1) A principal component (the blood vessel score) obtained by reducing the variables indicating the capillary condition and/or the non-capillary blood vessel condition by principal component analysis, or
   (2) a variable itself indicating the capillary condition and/or the non-capillary blood vessel condition (that is, the capillary condition per se and/or the non-capillary blood vessel condition per se) was used as the objective variable of the regression model.
   (1) In Case of Principal Component (Blood Vessel Score) Obtained by Reducing Variables Indicating Capillary Condition and/or Non-Capillary Blood Vessel Condition by Principal Component Analysis

As described above, data obtained by the OCT were subjected to principal component analysis, and the principal component calculated by the principal component analysis was used as the objective variable of the regression model. Specifically, image analysis was performed on images captured by the OCT to evaluate the three-dimensional structure of the vascular network in the dermis. Subsequently, quantitative analysis of the vascular network in the dermis was performed (the quantitative items were (1) the density of blood vessels, (2) the number of the blood vessels (the number of capillaries and the number of large blood vessels), (3) the number of branches of the blood vessels, (4) the distance from the base of the epidermis to the blood vessels, and (5) the thickness of the epidermis), and the quantitative values of the blood vessels were reduced by the principal component analysis, and the relationship between blood vessels and the skin was analyzed.

As described above, the principal component 1 (PC1) generated by the principal component analysis was positively correlated with “the number of capillaries”, “the density of blood vessels”, “the number of branches of the blood vessels”, and “the thickness of the epidermis”. The PC1 was negatively correlated with “the distance from the base of the epidermis to blood vessels”. In the present invention, the PC1 was used as the objective variable of the regression model. (The PC1 indicates how tightly the small blood vessels are networked. The PC1 is also referred to as the blood vessel score.) The principal component 2 (PC2) was positively correlated with “the number of large blood vessels”.

(2) In Case of Variable Itself Indicating Capillary Condition and/or Non-Capillary Blood Vessel Condition (That Is, Capillary Condition Per Se and/or Non-Capillary Blood Vessel Condition Per Se)

The variable itself indicating the capillary condition and/or the non-capillary blood vessel condition (specifically, at least one of the number of capillaries, the number of large blood vessels, the density of the blood vessels, the number of branches of the blood vessels, the distance from the base of the epidermis to the blood vessels, and the thickness of the epidermis) was used as the objective variable of the regression model.

### Generation of Model

A regression model for estimating “(1) a principal component (the blood vessel score) obtained by reducing the variables indicating the capillary condition and/or the non-capillary blood vessel condition by principal component analysis” or “(2) a variable itself indicating the capillary condition and/or the non-capillary blood vessel condition (that is, the capillary condition per se and/or the non-capillary blood vessel condition per se)” was examined using multiple regression analysis. Specifically, combinations of explanatory variables were examined in a brute-force manner to search for significant explanatory variables (the skin index of the face and the internal index related to the skin).

### Measurement of Skin Index and Internal Index of Face

First, the values of the following measurement items were measured for the following subjects. All measurement values were standardized (mean: 0, variance: 1).

### Subjects

gender: female/male, number of subjects: 274, age: 20 s-70 s

### Measurement Items:

age, moisture content, transepidermal water loss (TEWL), collagen density, wrinkle amount, firmness index, sagging degree, unevenness of skin texture, skin furrow area ratio, pore area ratio, skin tone (redness and yellowness), blemish amount, inflammatory cytokines (including cytokines collected from the horny layer, and referring to the amount of IL-1α, the amount of IL-1ra, the ratio of IL-1α to IL-1ra, the amount of SCCA1, the amount of S100A8A9, and the like), AGEs amount, and blood pressure

“(1) The principal component (the blood vessel score) obtained by reducing the variables indicating the capillary condition and/or the non-capillary blood vessel condition by principal component analysis”, and “(2) the variable itself indicating the capillary condition and/or the non-capillary blood vessel condition (that is, the capillary condition per se and/or the non-capillary blood vessel condition per se)” were calculated for the above subjects.

A multiple regression analysis was performed on the relationship between the skin index of the face and the capillary condition and/or the non-capillary blood vessel condition, and it was possible to estimate from the skin index of the face “(1) the principal component (the blood vessel score) obtained by reducing the variables indicating the capillary condition and/or the non-capillary blood vessel condition by principal component analysis”.

A multiple regression analysis was performed on the relationship between the skin index of the face and the capillary condition and/or the non-capillary blood vessel condition, and it was possible to estimate from the skin index of the face “(2) the variable itself indicating the capillary condition and/or the non-capillary blood vessel condition”. It was found that the capillary condition itself and/or the non-capillary blood vessel condition itself could be estimated using blood pressure as the internal index related to the skin of the face.

### Effect

In this manner, by focusing on blood vessels, it is possible to provide counseling and propose products and services related to beauty that focus on skin potential. For example, in counseling at a cosmetic store or at an event, it is possible to diagnose the capillary condition by extracting the skin index from a face image captured by a smart mirror, and to present skin characteristics based on the capillary condition. Furthermore, it is possible to suggest cosmetics that focus on the skin characteristics depending on the capillary condition.

### Hardware Configuration

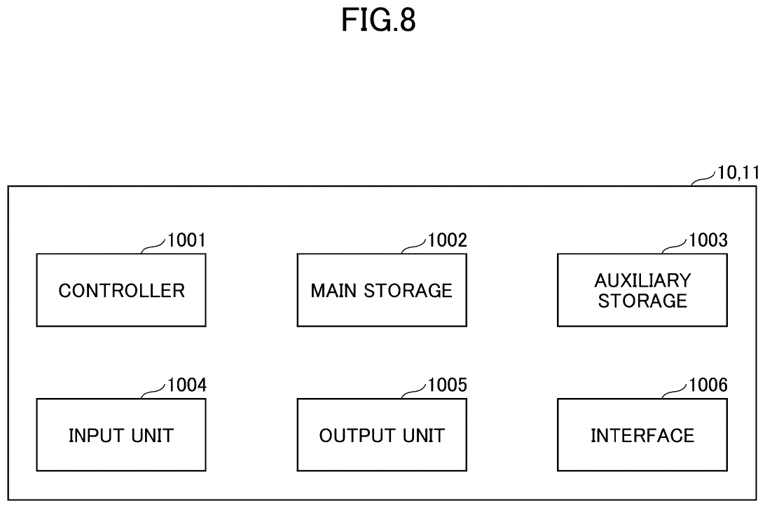

FIG. 8 is a hardware configuration diagram of the blood vessel estimation device 10 and the terminal 11 according to an embodiment of the present invention. The blood vessel estimation device 10 and the terminal 11 may include a controller 1001, a main storage 1002, an auxiliary storage 1003, an input unit 1004, an output unit 1005, and an interface 1006. Each of them will be described below.

The controller 1001 is a processor (for example, a central processing unit (CPU), a graphics processing unit (GPU), and the like) that executes various programs installed in the auxiliary storage 1003.

The main storage 1002 includes a non-volatile memory (a read only memory (ROM) ) and a volatile memory (a random access memory (RAM) ). The ROM stores various programs and data necessary for the controller 1001 to execute various programs installed in the auxiliary storage 1003. The RAM provides a work area into which the various programs installed in the auxiliary storage 1003 are expanded when loaded by the controller 1001.

The auxiliary storage 1003 is an auxiliary storage device that stores various programs and information used when the various programs are executed.

The input unit 1004 is an input device in which an operator of the blood vessel estimation device 10 and the terminal 11 inputs various instructions to the blood vessel estimation device 10 and the terminal 11.

The output unit 1005 is an output device that outputs the internal condition or the like of the blood vessel estimation device 10 and the terminal 11.

The interface 1006 is a communication device for connecting to a network and communicating with other devices.

Although the embodiments of the present invention have been described in detail above, the present invention is not limited to the specific embodiments described above, and various changes and modifications can be made within the scope of the present invention described in the claims.

The present international application claims priority to Japanese Patent Application No. 2022-166887 filed Oct. 18, 2022, the entire contents of which are incorporated herein by reference.

### DESCRIPTION OF REFERENCE NUMERALS

1 Blood vessel estimation system
10 Blood vessel estimation device
11 Terminal
20 Skin index measuring device
30 Subject
101 Acquisition unit
102 Blood vessel estimation unit
103 Skin estimation unit
104 Proposal unit
105 Model storage
106 Product information storage
1001 Controller
1002 Main storage
1003 Auxiliary storage
1004 Input unit
1005 Output unit
1006 Interface

1. A blood vessel estimation device comprising:
a processor; and
a memory storing a program, which when executed, causes the processor to:
acquire at least one of a skin index of a face or an internal index related to a skin of a subject; and
estimate at least one of a capillary condition or a non-capillary blood vessel condition of the face of the subject from the at least one of the skin index of the face or the internal index related to the skin of the subject.

2. The blood vessel estimation device according to claim 1, wherein the at least one of the capillary condition or the non-capillary blood vessel condition is a principal component obtained by reducing variables indicating the at least one of the capillary condition or the non-capillary blood vessel condition by principal component analysis.

3. The blood vessel estimation device according to claim 2, wherein the principal component correlates with at least one of: a number of capillaries, a density of blood vessels, a number of branches of the blood vessels, a distance from a base of an epidermis to the blood vessels, or a thickness of the epidermis.

4. The blood vessel estimation device according to claim 1, wherein the at least one of the capillary condition or the non-capillary blood vessel condition is at least one of: (1) a density of blood vessels, (2) a number of the blood vessels (3) a number of branches of the blood vessels, (4) a distance from a base of an epidermis to the blood vessels, or (5) a thickness of the epidermis.

5. The blood vessel estimation device according to claim 1, wherein the skin index is an index related to skin aging.

6. The blood vessel estimation device according to claim 1, wherein the skin index is an index related to skin roughness.

7. The blood vessel estimation device according to claim 1, wherein the skin index is at least one of: a blemish amount, transepidermal water loss (TEWL), redness, yellowness, a skin furrow area ratio, an unevenness of skin texture, a pore area ratio, collagen density, a wrinkle amount, a firmness index, a sagging degree, or inflammatory cytokines.

8. The blood vessel estimation device according to claim 1, wherein the internal index is blood pressure.

9. The blood vessel estimation device according to claim 1, wherein the skin index is acquired from an image in which the face of the subject is captured.

10. The blood vessel estimation device according to claim 1 wherein the program further causes the processor to estimate which of a plurality of types a future skin condition of the subject is, from the at least one of the capillary condition or the non-capillary blood vessel condition that is estimated.

11. The blood vessel estimation device according to claim 10, wherein the program further causes the processor to propose at least one of products or services related to beauty according to at least one of: the at least one of the capillary condition or the non-capillary blood vessel condition; or the future skin condition.

12. The blood vessel estimation device according to claim 1, wherein the program causes the processor to estimate the at least one of the capillary condition or the non-capillary blood vessel condition of the face of the subject from the at least one of the skin index of the face or the internal index related to the skin of the subject by using a regression model.

13. The blood vessel estimation device according to claim 12, wherein the regression model is a statistical model or a machine learning model.

14. A blood vessel estimation method comprising:
acquiring at least one of a skin index of a face or an internal index related to a skin of a subject; and
estimating at least one of a capillary condition or a non-capillary blood vessel condition of the face of the subject from the at least one of the skin index of the face or the internal index related to the skin of the subject.

15. A non-transitory computer-readable recording medium storing a program, which when executed, causes a processor to:
acquire at least one of a skin index of a face or an internal index related to a skin of a subject; and
estimate at least one of a capillary condition or a non-capillary blood vessel condition of the face of the subject from the at least one of the skin index of the face or the internal index related to the skin of the subject.
